(19) United States
(12) Patent Application Publication
(10) Pub. No.: US 2022/0106187 A1
(43) Pub. Date: Apr. 7, 2022

(54) STABLE LIPID BILAYERS ON NANOPORE ARRAYS

(71) Applicant: Quantapore, Inc., South San Francisco, CA (US)

(72) Inventor: Ossama Assad, Redwood City, CA (US)

(73) Assignee: Quantapore, Inc., South San Francisco, CA (US)

(21) Appl. No.: 17/443,017

(22) Filed: Jul. 19, 2021

Related U.S. Application Data
(63) Continuation of application No. 16/615,092, filed on Nov. 19, 2019, now abandoned.
(60) Provisional application No. 62/511,484, filed on May 26, 2017.

(51) Int. Cl. B81C 1/00; B82Y 15/00; B82Y 40/00; C12Q 1/6869; G01N 33/487

(52) U.S. Cl. CPC B81C 1/00087; B82Y 15/00; B82Y 40/00; B81C 2201/0133; G01N 33/48721; B81C 2201/0132; C12Q 1/6869

(57) ABSTRACT

The invention is directed to methods of making stable lipid bilayers supported by a solid state nanopore array. Exemplary methods include the steps of masking a first layer on a planar support to form dry etch zones; dry etching the dry etch zones to form an array of apertures extending into but not through the first layer; masking a second side of the planar support body to form an etch region aligned with the array of apertures; wet etching the etch region to expose a surface of the first layer; dry etching the exposed surface of the first layer to a depth overlapping the apertures so that apertures of the array provide fluid communication across the first layer; and disposing a lipid bilayer on a surface of the first layer on a side opposite the planar support which encompasses the array of apertures.

### CROSS-REFERENCE TO RELATED APPLICATIONS

This application is a continuation of U.S. patent application Ser. No. 16/615,092, filed Nov. 19, 2019, which is a U.S. national application filed under 35 U.S.C. 371 of PCT International Application No. PCT/US2018/030708, filed May 2, 2018, which claims priority to U.S. Patent Application No. 62/511,484 filed May 26, 2017, the content of each of which is incorporated herein by reference in its entirety.

### BACKGROUND

A promising class of biosensors have been developed which employ as detection elements membrane proteins inserted into a lipid bilayer supported by a nanopore array. Membrane proteins may play either functional roles, such as analyte recognition and signal transduction, and/or structural roles, such as a conduit with a precise geometry for moving ions or analytes across the bilayer in a detection process. A major challenge in the application of such biosensors has been developing methods for conveniently producing lipid bilayers that are sufficiently stable and rugged to permit measurements over a useful interval of time.

The development of biosensors using supported lipid bilayers, such as those used in single molecule analysis, would be advanced by the availability of methods for producing lipid bilayers disposed on nanopore arrays, which have improved stability.

### SUMMARY OF THE INVENTION

The present invention is directed to methods for making devices and/or articles of manufacture comprising a lipid bilayer supported by a solid state nanopore array. In some embodiments, the invention further includes methods of making precursor articles wherein the solid state nanopore array includes a reflective surface.

In some embodiments, methods of the invention comprise the following steps: (a) disposing a first layer of known thickness on a first side of a planar support body; (b) masking the first layer to form an array of dry etch zones; (c) dry etching the dry etch zones to form an array of apertures (or nanopores) extending into but not through the first layer; (d) masking a second side of the planar support body to form an etch region aligned with the array of apertures (or nanopores); (e) wet etching the etch region on the second side of the planar support body to expose a surface of the first layer; and (f) dry etching the exposed surface of the first layer to a depth overlapping the apertures so that apertures of the array provide fluid communication across the first layer to produce a solid state nanopore array. In some embodiments, methods of the invention include a further step of disposing a lipid bilayer on a surface of the first layer on a side opposite the planar support body.

The present invention is exemplified in a number of implementations and applications, some of which are summarized below and throughout the specification.

### BRIEF DESCRIPTION OF THE DRAWINGS

FIGS. 1A-1B illustrate a problem the invention addresses.

FIGS. 2A-2C illustrate an embodiment of the invention.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention. Guidance for aspects of the invention is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2^{nd} edition (Wiley Interscience, 1995); Bard and Faulkner, Electrochemical Methods: Fundamentals and Applications, 2^{nd} edition (Wiley, 2000); Lakowicz, Principles of Fluorescence Spectroscopy, 3^{rd} edition (Springer, 2006); Hermanson, Bioconjugate Techniques, Second Edition (Academic Press, 2008); and the like, which relevant parts are hereby incorporated by reference.

In one aspect, the invention is directed to methods of making stable lipid bilayers supported by a solid state nanopore array. In some embodiments, the invention improves stability of lipid bilayers by providing solid state nanopore arrays without surface damage caused by exposure to wet etchants used in their manufacture. The invention also includes the applications of such supported bilayers in devices for single molecule analysis, such as, nucleic acid sequencing, and the like. In part, the invention is a recognition and appreciation that the manner in which the solid state nanopore array is fabricated has a significant effect on lipid bilayer stability and blockage of solid state nanopores. In some embodiments, methods of the invention provide a fabrication method that reduces the presence of wet etching debris on the surface of the solid state membrane accepting the lipid bilayer, thereby reducing blocked nanopores and surface debris that disrupts bilayer stability.

Exemplary methods include the steps of masking a first layer, e.g. of silicon nitride, on a planar support, e.g. silicon, to form dry etch zones; dry etching the dry etch zones to form an array of apertures extending into but not through the first layer; masking a second side of the planar support body to form an etch region aligned with the array of apertures; wet etching the etch region to expose a surface of the first layer; dry etching the exposed surface of the first layer to a depth overlapping the apertures so that apertures of the array provide fluid communication across the first layer; and disposing a lipid bilayer on a surface of the first layer on a side opposite the planar support which encompasses the array of apertures.

In some embodiments, the invention includes methods of making solid state nanopore arrays which may be used to support lipid bilayers and which comprise a metal layer, such as, a layer of aluminum, silver or gold, or especially, a layer of aluminum. In such embodiments, in addition to preventing surface debris and nanopore blockage, the method of the invention prevents damage to the metal layer.

In some embodiments, methods of the invention include the following steps: (a) disposing a first layer of known thickness on a first side of a planar support body; (b) masking the first layer to form an array of dry etch zones; (c) dry etching the dry etch zones to form an array of apertures (or nanopores) extending into but not through the first layer; (d) masking a second side of the planar support body to form an etch region aligned with the array of apertures (or nanopores); (e) wet etching the etch region on the second side of the planar support body to expose a surface of the first layer; and (f) dry etching the exposed surface of the first layer to a depth overlapping the apertures so that apertures of the array provide fluid communication across the first layer to produce a solid state nanopore array. The array of apertures contains a plurality of apertures which may be arranged in a wide variety of patterns. In some embodiments, the array comprises a number of apertures in the range of from 2 to 1000; in other embodiments, an array comprises a number of apertures in the range of from 2 to 100. In some embodiments, the array of apertures is a rectilinear array; in some embodiments, the array of apertures is a square array; in some embodiments, the array of apertures is a hexagonal array. In some embodiments, methods of the invention further include the step of disposing a lipid bilayer on a surface of the first layer on a side opposite the planar support body which encompasses the array of apertures. In some embodiments, the first layer comprises a plurality of layers wherein the distal most layer (or outer most layer) of the plurality from the planar support body is a metal layer. In some embodiments, the first layer comprises two layers. In some embodiments, the first layer comprises a layer of silicon nitride on the planar support body and a layer of aluminum on the silicon nitride opposite of the planar support body. In some embodiments, the planar support layer is silicon and the steps of wet etching are carried out by silicon etchants.

Wet etchants for carrying out wet etching steps of the invention comprise an oxidizer, an acid or base to dissolve an oxidized surface created by the oxidizer, and a solvent or dilutent media to transport reactants and products. Exemplary oxidants include hydroxides, such as KOH, NaOH, CeOH, RbOH, NH₄OH, TMAH (tetramethylammonium hydroxide), (CH3)4NOH, and the like. Exemplary solvents are water and acetic acid.

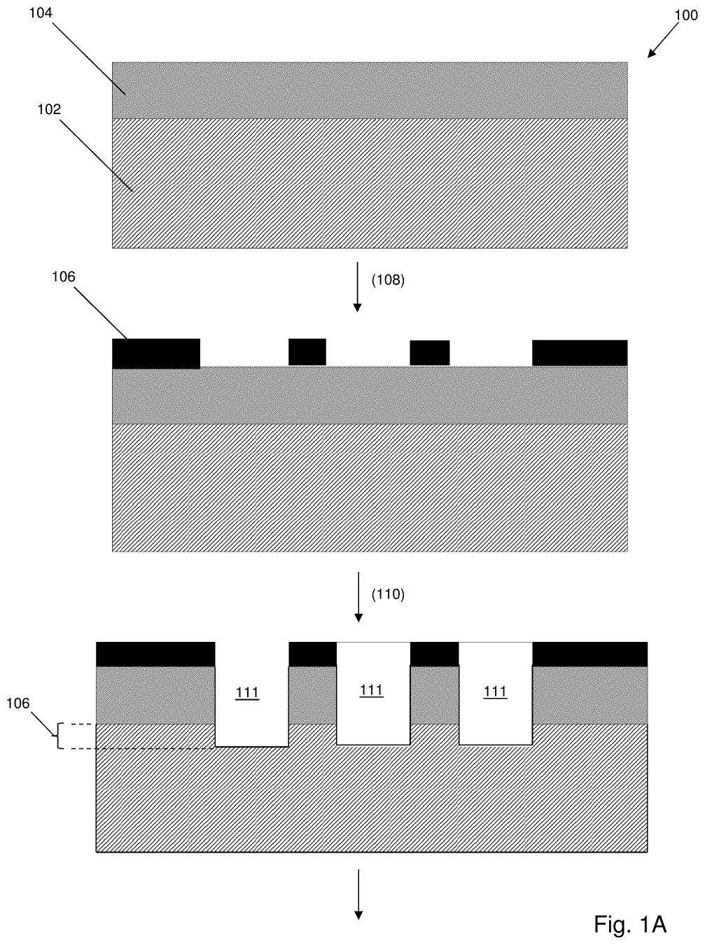

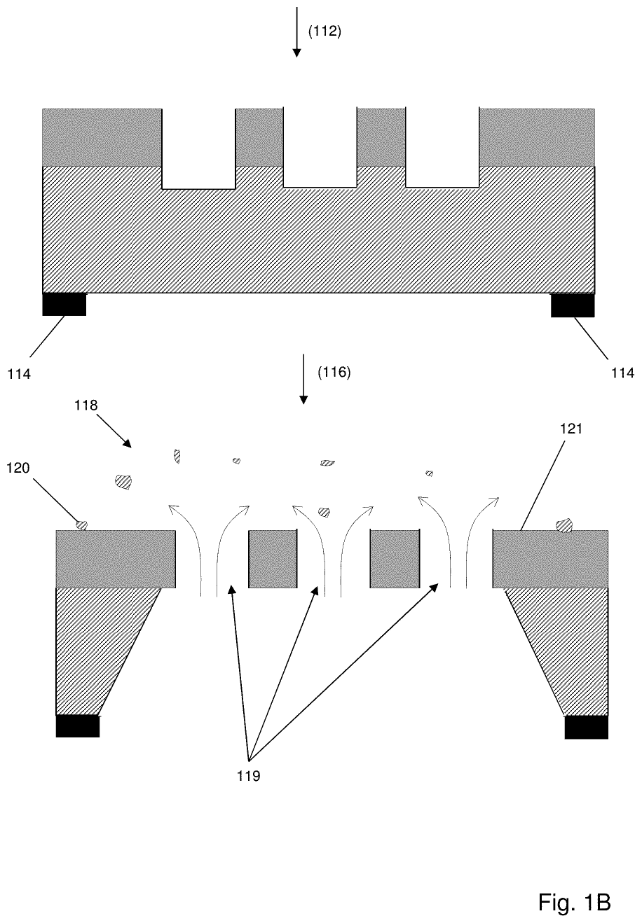

FIG. 1A-1B illustrate concepts related to prior art methods. Solid state membrane (100) comprising silicon wafer (102) with first layer (104), such as silicon nitride, is patterned (108) with a photoresist (106) for dry etching of wells (111) using conventional masking and dry etching techniques. Wells (111), for example, may have cross-sectional dimensions in the range of 1-1000 nm diameter (for circular cross section) or 1-1000 nm widths (for square cross section); or in some embodiments, wells (111) have cross-sectional dimensions in the range of 10-100 nm diameter (for circular cross section) or 10-100 nm widths (for square cross section). Typically to form nanopores from such wells the dry etching produces a passage completely through layer (104) and partially into layer (102), for example, by a depth (106), after which (as illustrated in FIG. 1B) the reverse side of solid state membrane (100) is masked (112) with photoresist (114) so that material from layer (102) can be removed by wet etching (116, e.g. with KOH) down to layer (104) to reveal nanopores (119). Unfortunately, such a wet etching step releases material (118 & 120) that deposits on surface (121) or lodges in nanopores, which result in solid state membranes that cannot stably support lipid bilayers or that are otherwise inefficient because a percentage of nanopores of the array are blocked wholly or partially. Furthermore, whenever first layer (104) comprises a plurality of sub-layers one of which is a metal layer, such as an aluminum layer, wet etching with an etchant like KOH will damage the aluminum layer.

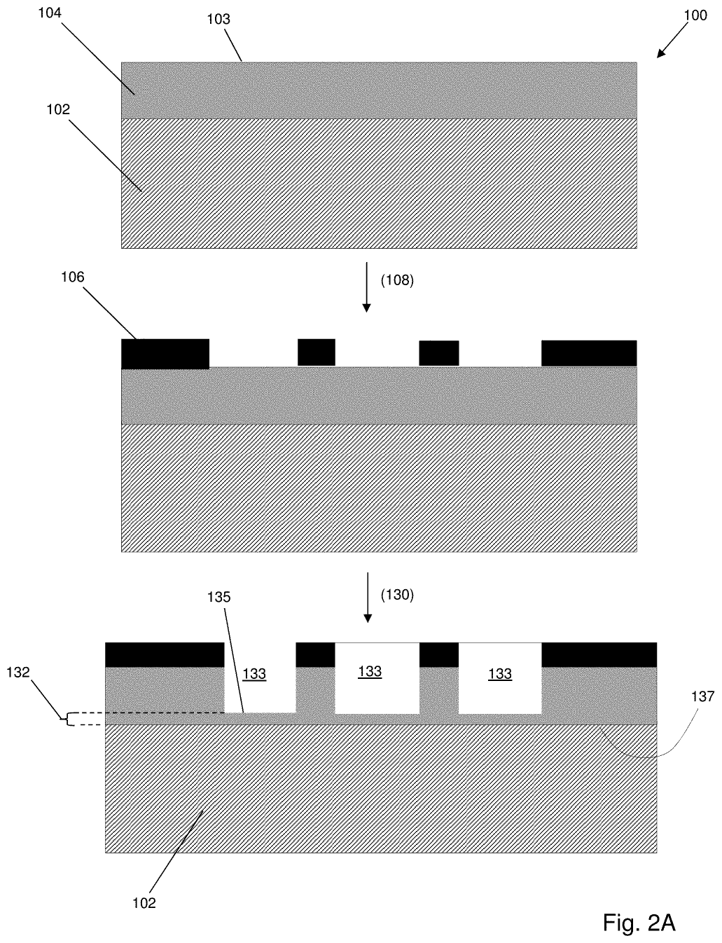

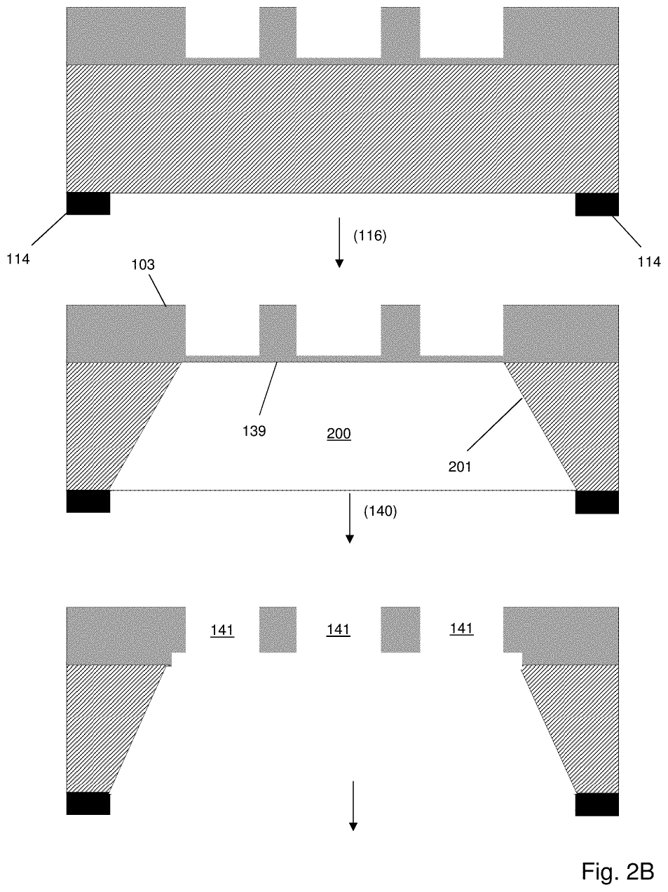

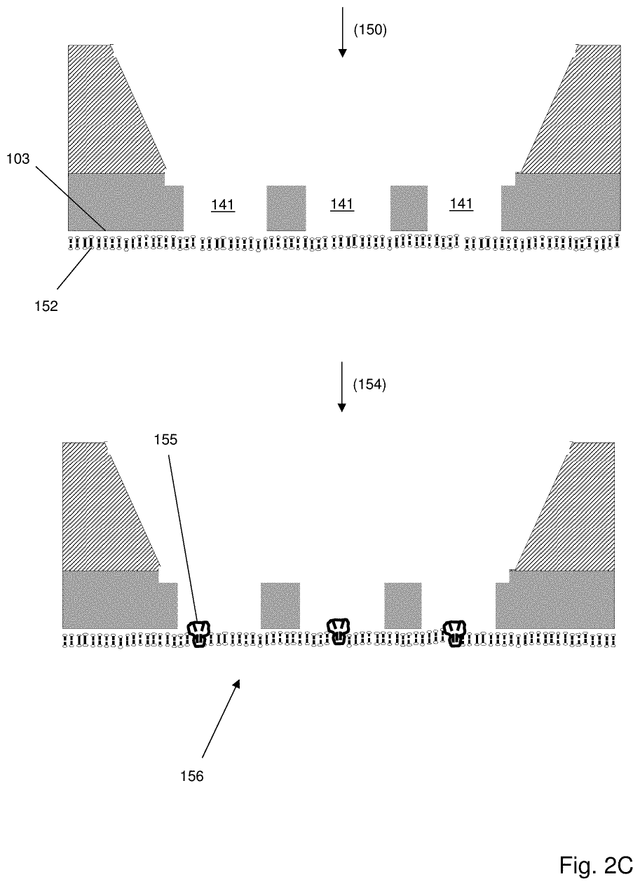

FIGS. 2A-2C illustrate formation of a supported bilayer by a method of the invention which overcomes the above deficiency of the prior art. Surface (103) of first layer (104) is masked (108) with photoresist (106) as above to form dry etch zones, after which solid state membrane (100) is dry etched (130) to form wells (133) whose bottoms or floors (135) do not extend into the material of layer (102); that is, wells (133) are confined entirely to layer (104), leaving a predetermined thickness (132) between bottoms (135) and surface (137) of layer (102). In some embodiments, layer (102) is a silicon wafer having a thickness in the range of from 200-1000 μm and first layer (104) is silicon nitride having a thickness in the range of from 20 to 200 nm, which may be formed using chemical vapor deposition, atomic layer deposition, LPCVD, physical vapor deposition, or like technique. In some embodiments, layer (104) comprises two layers (or sub-layers), one sub-layer of silicon nitride adjacent to layer (102) and one sub-layer of a metal, such as aluminum, on the surface of the silicon nitride opposite to layer (102). In some embodiments, such predetermined thickness (132) may be in the range of from 2 to 30 nm, particularly when layer (104) comprises silicon nitride or a sub-layer adjacent to layer (102) comprises silicon nitride. As above, the reverse side of solid state membrane (100) is masked with photoresist (114) to form an etch region aligned with wells (133) and wet etched to surface (139) of layer (104); however, because the wet etching does not open passages to surface (103) of layer (104) no debris is deposited thereon nor can debris lodge in wells (133). In some embodiments, the etch region aligned with wells (133) defines an area whose center corresponds to the center of the collections of wells (133). Thus, in some embodiments where the collection of wells (133) forms a rectilinear array of wells, the center of the rectilinear array and the center of the etch region lie on the same line perpendicular to solid state membrane (100). Typically, the etch region covers an area larger than and encompassing the array of wells (133) so that after wet etching creates trench (200) with angular walls (201) its floor or surface (139) still encompasses the collection or array of wells (133). Photoresist mask (114) is designed so that the removal of material in the wet etching step exposes an area of surface (137) that encompasses all the nanopores (133) of the array. Dry etching step (140) is then employed to remove material from bottoms of wells (133) to form nanopores (141) without production of undesirable debris. After an optional washing step, lipid bilayer (152) is disposed (150) on surface (103) so that it spans nanopores (141), after which (or concurrently with) membrane proteins (155) may be inserted (154) into lipid bilayer (152) to form device (156).

First layer (104) may comprise a single material or it may comprise a plurality of sub-layers. In some embodiments, at least one sub-layer of layer (104) is a metal oxide or a nitride, such as SiO₂, Al₂O₃, SiNₓ, HfO₂, TiO₂, silica, and the like. In some embodiments, layer (104) is silicon nitride or silicon oxide. In still other embodiments, layer (104) comprises a plurality of sub-layers, at least one of which is a metal layer. Layer (102) may comprise a wide range of MEMS materials including, but not limited to, silicon, silicon nitride, silicon dioxide, and the like. In some embodiments, layer (102) is silicon.

Surfaces of solid state membrane (100) may be masked using conventional photoresists and masking techniques, and particular embodiments of the invention may include optional photoresist stripping or removal steps. For example, photoresist removal after dry etching may include treatment with organic solvents, piranha solution, or treatment by “aching” remaining photoresist material. Exemplary piranha solutions comprise sulfuric acid (H2SO4) and hydrogen peroxide (H2O2) mixture. The ratio of H2SO4:H2O2 may vary, but commonly a mixture by volume of between 2:1 and 4:1 H2SO4:(96 wt %):H2O2 (30 wt %), ratios as high as 8:1.

### Solid State Membranes, Apertures and Nanopores

Important features of nanopores include constraining polynucleotide analytes, such as labeled polynucleotides so that their monomers pass through a signal generation region (or equivalently, an excitation zone, or detection zone, or the like) in sequence. That is, a nanopore constrains the movement of a polynucleotide analyte, such as a polynucleotide, so that nucleotides pass through a detection zone (or excitation region) in single file. In some embodiments, additional functions of nanopores include (i) passing single stranded nucleic acids while not passing double stranded nucleic acids, or equivalently bulky molecules and/or (ii) constraining fluorescent labels on nucleotides so that fluorescent signal generation is suppressed or directed so that it is not collected.

In some embodiments, nanopores used in connection with the methods and devices of the invention are provided in the form of arrays, such as an array of clusters of nanopores, which may be disposed regularly on a planar surface. In some embodiments, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

Solid state membranes with apertures (sometime referred to as “solid state nanopores”) may be fabricated in a variety of materials including but not limited to, silicon, amorphous silicon, and metal oxide and nitrides, including SiO₂, Al₂O₃, SiNₓ, HfO₂, TiO₂, silica, and the like. The fabrication and operation of solid state nanopores for analytical applications, such as DNA sequencing, are disclosed in the following exemplary references that are incorporated by reference: Ling, U.S. Pat. No. 7,678,562; Hu et al, U.S. Pat. No. 7,397,232; Golovchenko et al, U.S. Pat. No. 6,464,842; Chu et al, U.S. Pat. No. 5,798,042; Sauer et al, U.S. Pat. No. 7,001,792; Su et al, U.S. Pat. No. 7,744,816; Church et al, U.S. Pat. No. 5,795,782; Bayley et al, U.S. Pat. No. 6,426,231; Akeson et al, U.S. Pat. No. 7,189,503; Bayley et al, U.S. Pat. No. 6,916,665; Akeson et al, U.S. Pat. No. 6,267,872; Meller et al, U.S. patent publication 2009/0029477; Howorka et al, International patent publication WO2009/007743; Brown et al, International patent publication WO2011/067559; Meller et al, International patent publication WO2009/020682; Polonsky et al, International patent publication WO2008/092760; Van der Zaag et al, International patent publication WO2010/007537; Yan et al, Nano Letters, 5(6): 1129-1134 (2005); Iqbal et al, Nature Nanotechnology, 2: 243-248 (2007); Wanunu et al, Nano Letters, 7(6): 1580-1585 (2007); Dekker, Nature Nanotechnology, 2: 209-215 (2007); Storm et al, Nature Materials, 2: 537-540 (2003); Wu et al, Electrophoresis, 29(13): 2754-2759 (2008); Nakane et al, Electrophoresis, 23: 2592-2601 (2002); Zhe et al, J. Micromech. Microeng., 17: 304-313 (2007); Henriquez et al, The Analyst, 129: 478-482 (2004); Jagtiani et al, J. Micromech. Microeng., 16: 1530-1539 (2006); Nakane et al, J. Phys. Condens. Matter, 15 R1365-R1393 (2003); DeBlois et al, Rev. Sci. Instruments, 41(7): 909-916 (1970); Clarke et al, Nature Nanotechnology, 4(4): 265-270 (2009); Bayley et al, U.S. patent publication 2003/0215881; and the like.

In some embodiments, solid state membranes with apertures or nanopores are fabricated with conventional wet etching and/or dry etching processes. Wet etching includes immersion or spray etching. Dry etching includes plasma etching, reactive ion etching or sputter etching.

In some embodiments, the invention comprises nanopore arrays with one or more light-blocking layers, that is, one or more opaque layers. Typically nanopore arrays are fabricated in thin sheets of material, such as, silicon, silicon nitride, silicon oxide, aluminum oxide, or the like, which readily transmit light, particularly at the thicknesses used, e.g. less than 50-100 nm. For electrical detection of analytes this is not a problem. However, in optically-based detection of labeled molecules translocating nanopores, light transmitted through an array invariably excites materials outside of intended reaction sites, thus generates optical noise, for example, from nonspecific background fluorescence (such as silicon nitride photoluminescence), fluorescence from labels of molecules that have not yet entered a nanopore, or the like. In one aspect, the invention addresses this problem by providing nanopore arrays with one or more light-blocking layers that reflect and/or absorb light from an excitation beam, thereby reducing background noise for optical signals generated at intended reaction sites associated with nanopores of an array. In some embodiments, this permits optical labels in intended reaction sites to be excited by direct illumination. In some embodiments, an opaque layer may be a metal layer. Such metal layer may comprise Sn, Al, V, Ti, Ni, Mo, Ta, W, Au, Ag or Cu, or a plurality of sub-layers of different selections such metals. In some embodiments such metal layer may comprise Al, Au, Ag or Cu, or a plurality of sub-layers of different selections of such metals. In still other embodiments, such metal layer may comprise aluminum or gold, or may comprise solely aluminum. The thickness of an opaque layer may vary widely and depends on the physical and chemical properties of material composing the layer. In some embodiments, the thickness of an opaque layer may be at least 5 nm, or at least 10 nm, or at least 40 nm. In other embodiments, the thickness of an opaque layer may be in the range of from 5-100 nm; in other embodiments, the thickness of an opaque layer may be in the range of from 10-80 nm. An opaque layer need not block (i.e. reflect or absorb) 100 percent of the light from an excitation beam. In some embodiments, an opaque layer may block at least 10 percent of incident light from an excitation beam; in other embodiments, an opaque layer may block at least 50 percent of incident light from an excitation beam.

Opaque layers or coatings may be fabricated on solid state membranes by a variety of techniques known in the art. Material deposition techniques may be used including chemical vapor deposition, electrodeposition, epitaxy, thermal oxidation, physical vapor deposition, including evaporation and sputtering, casting, and the like. In some embodiments, atomic layer deposition may be used, e.g. U.S. Pat. No. 6,464,842; Wei et al, Small, 6(13): 1406-1414 (2010), which are incorporated by reference.

In some embodiments, a 1-100 nm channel or aperture may be formed through a solid substrate, usually a planar substrate, such as a membrane, through which an analyte, such as single stranded DNA, is induced to translocate. In other embodiments, a 2-50 nm channel or aperture is formed through a substrate; and in still other embodiments, a 2-30 nm, or a 2-20 nm, or a 3-30 nm, or a 3-20 nm, or a 3-10 nm channel or aperture if formed through a substrate.

In some embodiments, methods and devices of the invention comprise a solid phase membrane, such as a silicon nitride membrane, having an array of apertures therethrough providing communication between a first chamber and a second chamber (also sometimes referred to as a “cis chamber” and a “trans chamber”). In some embodiments, devices of the invention comprise such solid phase membranes and a lipid bilayer disposed on a surface of the solid phase membrane. In some embodiments, diameters of the aperture in such a solid phase membrane may be in the range of 10 to 200 nm, or in the range of 20 to 100 nm. In some embodiments, such solid phase membranes further include protein nanopores inserted into the lipid bilayer in regions where such bilayer spans the apertures on the surface facing the trans chamber. In some embodiments, such protein nanopores are inserted from the cis side of the solid phase membrane using techniques described herein.

### Bilayer Deposition

The step of disposing a lipid bilayer on a surface of a solid state membrane prepared in accordance with the invention can be carried out in a variety of ways including, but not limited to painting, Muller-Montal method or by way of unilamellar vesicles, e.g. Studer, Doctoral Thesis ETH No. 18473 (ETH Zurich, 2009). Of particular interest is the deposition of lipid bilayers by unilamellar vesicles, for example as disclosed by the following references: Urban et al, Nano Letters, 14: 1674-1680 (2014); Im et al, Chemical Science, 1: 688-696 (2010); Kleefen et al, Nano Letters, 10: 5080-5087 (2010); Kumar et al, Langmuir, 27: 10920-10928 (2011); and the like.

Stability of a lipid bilayer on a solid state nanopore array depends on several factors including the chemical nature of the support surface, the nature of the lipids, presence or absence of surface defects and/or debris, the size and number of nanopores, and the like. Stability may be determined using a variety of techniques including measurement of resistance across the array, measurement of impedance across the array (e.g. by impedance spectroscopy), measurement of capacitance across the array, as well as by characterization of the surface of an array by atomic force microscopy, and other imaging techniques, such as confocal microscopy, STED, or the like. Such techniques are disclosed in the following references: Studer, Doctoral Thesis (cited above); Kumar et al (cited above); Dufrene et al, Biochimica et Biophysica Acta, 1509: 14-41 (2000); Jass et al, Biophysical J., 79: 3153-3163 (2000); or the like. A measure of stability that is particularly useful is the change over a given time interval of values of a measurement obtained from one or more of the techniques listed above. A change in the value for impedance across an array after an interval of time is of particular interest. In some embodiments of the invention, impedance across an array is at least 1 Giga-ohm and such an initially measured value is maintained on average for at least 1 hour from the time a bilayer is deposited on a surface of the solid state nanopore array, particularly whenever such surface is silicon nitride or aluminum. In some embodiments, supported lipid bilayers of the invention are at least twice as likely to maintain at least 1 Giga-ohm resistance after 1 hour than those made using a single dry etch method, wherein the surface of the first layer is exposed to wet etchant compounds. In some embodiments of the invention, impedance across an array is at least 1 Giga-ohm and such an initially measured value is maintained on average for at least 4 hours from the time a bilayer is deposited on a surface of the solid state nanopore array, particularly whenever such surface is silicon nitride or aluminum. In some embodiments, supported lipid bilayers of the invention are at least twice as likely to maintain at least 1 Giga-ohm resistance after 4 hours than those made using a single dry etch method, wherein the surface of the first layer is exposed to wet etchant compounds. In some embodiments of the invention, impedance across an array is at least 1 Giga-ohm and such an initially measured value is maintained on average for at least 8 hours from the time a bilayer is deposited on a surface of the solid state nanopore array, particularly whenever such surface is silicon nitride or aluminum. In some embodiments, supported lipid bilayers of the invention are at least twice as likely to maintain at least 1 Giga-ohm resistance after 8 hours than those made using a single dry etch method, wherein the surface of the first layer is exposed to wet etchant compounds. In some embodiments of the invention, impedance across an array is at least 1 Giga-ohm and such an initially measured value is maintained on average for at least 24 hours from the time a bilayer is deposited on a surface of the solid state nanopore array, particularly whenever such surface is silicon nitride or aluminum. In some embodiments, supported lipid bilayers of the invention are at least twice as likely to maintain at least 1 Giga-ohm resistance after 24 hours than those made using a single dry etch method, wherein the surface of the first layer is exposed to wet etchant compounds. In some embodiments of the foregoing, nanopore arrays comprise from 9 to 10,000 nanopores each having a cross-sectional area (usually with circular geometry) of from 3 to 1.2×10⁴ nm² and spaced regularly within an area less than 2 cm². In some embodiments of the foregoing, nanopore arrays comprise from 9 to 1000 nanopores each having a cross-sectional area (usually with circular geometry) of from 3 to 1.2×10⁴ nm² and spaced regularly within an area less than 1 cm². In some embodiments of the foregoing, nanopore arrays comprise from 9 to 1000 nanopores each having cross-sectional areas (usually with circular geometry) of from 3 to 1.2×10⁴ nm² and spaced regularly within an area less than 0.25 cm². In some embodiments of the foregoing, nanopore arrays comprise from 9 to 1000 nanopores each having cross-sectional area (usually with circular geometry) of from 3 to 1.2×10⁴ nm² and spaced regularly within an area less than 10⁴ μm².

A wide variety of lipids may be used to form lipid bilayers on nanopore arrays. In some embodiments, lipid mixtures containing phosphatidylcholine (PC), phosphatidylethanolamine (PE), phosphatidylglycerol (PG), or cholesterol can be used.

By way of example, lipid membranes and their formation process on a nanopore array may be characterized by impedance spectroscopy using commercially available instruments, such as the gain/phase analyzer SI 1260 and the 1296 Dielectric Interface (Solartron Instruments, Farnborough, UK).

### Molecular Analysis Using Devices of the Invention

As mentioned above, devices made by methods of the invention may be used to analyze molecules by a variety of approaches including, but not limited to, electrical or optical signatures generated as a molecule of interest passes through the bore of a protein nanopore imbedded in a lipid bilayers of a device. Of particular interest is the analysis of single molecules by way of optical signatures they generate as they pass, or translocate, through the bore of a protein nanopore of the device. Such optical signatures may come from an analyte directly or from an optical label attached to the analyte, or both. In some embodiments, analytes detected by devices using a device of the invention include polynucleotides labeled with one of more optical labels, particularly two or more optical labels that generate distinguishable signals that permit nucleotides to which they are attached to be identified. That is, in some embodiments, articles of the invention are used in a device from determining a nucleotide sequence of a polynucleotide. Guidance for such applications is disclosed in the following references including, but not limited to, U.S. provisional patent application Ser. Nos. 62/308,145; 62/372,928; 62/322343; 62/421804; U.S. patent publications US2016/0076091; US2016/0122812; and the like, which references are incorporated herein by reference.

In some embodiments, a device for implementing the above methods for analyzing polynucleotides (such as single stranded polynucleotides) typically includes a set of electrodes for establishing an electric field across the layered membrane and nanopores. Single stranded nucleic acids are exposed to nanopores by placing them in an electrolyte in a first chamber, which is configured as the “cis” side of the layered membrane by placement of a negative electrode in the chamber. Upon application of an electric field, the negatively single stranded nucleic acids are captured by nanopores and translocated to a second chamber on the other side of the layered membrane, which is configured as the “trans” side of membrane by placement of a positive electrode in the chamber. The speed of translocation depends in part on the ionic strength of the electrolytes in the first and second chambers and the applied voltage across the nanopores. In optically based detection, a translocation speed may be selected by preliminary calibration measurements, for example, using predetermined standards of labeled single stranded nucleic acids that generate signals at different expected rates per nanopore for different voltages. Thus, for DNA sequencing applications, a translocation speed may be selected based on the signal rates from such calibration measurements. Consequently, from such measurements a voltage may be selected that permits, or maximizes, reliable nucleotide identifications, for example, over an array of nanopores. In some embodiments, such calibrations may be made using nucleic acids from the sample of templates being analyzed (instead of, or in addition to, predetermined standard sequences). In some embodiments, such calibrations may be carried out in real time during a sequencing run and the applied voltage may be modified in real time based on such measurements, for example, to maximize the acquisition of nucleotide-specific signals.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

What is claimed is:

1. A method of making a supported lipid bilayer comprising:
disposing a first layer of known thickness on a first side of a planar support body;
masking the first layer to form an array of dry etch zones;
dry etching the dry etch zones to form an array of apertures extending into but not through the first layer;
masking a second side of the planar support body to form an etch region aligned with the array of apertures;
wet etching the etch region on the second side of the planar support body to expose a surface of the first layer;
dry etching the exposed surface of the first layer to a depth overlapping the apertures so that apertures of the array provide fluid communication across the first layer; and
disposing a lipid bilayer on a surface of the first layer on a side opposite the planar support body which encompasses the array of apertures.

2. The method of claim 1 wherein said first is silicon nitride or silicon oxide and wherein said planar support body comprises silicon.

3. The method of claim 2 wherein an impedance across said array after said deposition of said lipid bilayer is at least 1 Giga-ohm for at least 4 hours and wherein said array comprises from 9 to 10,000 apertures each having a cross-sectional area of from 3 to 1.2×10⁴ nm² and spaced regularly within an area less than 2 cm².

4. A method of making a supported lipid bilayer comprising:
disposing a silicon nitride layer of known thickness on a first side of a planar body of silicon;
masking the silicon nitride layer to form an array of dry etch zones;
dry etching the dry etch zones to form an array of apertures extending into but not through the silicon nitride layer;
masking a second side of the planar body of silicon to form an etch region aligned with the array of apertures;
wet etching the etch region on the second side of the planar body of silicon to expose a surface of the silicon nitride layer;
dry etching the exposed surface of the silicon nitride layer to a depth overlapping the apertures so that apertures of the array provide fluid communication across the silicon nitride layer;
disposing a lipid bilayer on a surface of the silicon nitride layer on a side opposite the planar body of silicon which encompasses the array of apertures.

5. The method of claim 4 further including a step of washing said surface of silicon nitride prior to said step of disposing said lipid bilayer.

6. The method of claim 4 wherein an impedance across said array after said deposition of said lipid bilayer is at least 1 Giga-ohm for at least 4 hours and wherein said array comprises from 9 to 10,000 apertures each having a cross-sectional area of from 3 to 1.2×10⁴ nm² and spaced regularly within an area less than 2 cm².

7. A method of making a nanopore array having a metal surface, the method comprising the steps of:
disposing a first layer of known thickness on a first side of a planar support body, the first layer comprising a plurality of sub-layers including a metal sub-layer having an outer surface opposite of the planar support body;
masking the first layer to form an array of dry etch zones;
dry etching the dry etch zones to form an array of apertures extending into but not through the first layer;
masking a second side of the planar support body to form an etch region aligned with the array of apertures;
wet etching the etch region on the second side of the planar support body to expose a surface of the first layer; and
dry etching the exposed surface of the first layer to a depth overlapping the apertures so that apertures of the array provide fluid communication across the first layer to produce a nanopore array having a metal surface.

8. The method of claim 7 wherein said first layer comprises a sub-layer of silicon nitride on said planar support body and a sub-layer of aluminum on the sub-layer of silicon nitride.

9. The method of claim 8 wherein said planar support body comprises silicon.

10. The method of claim 9 further comprising a step of disposing a lipid bilayer on a surface of said first layer on a side opposite said planar support body which encompasses said array of apertures.

11. The method of claim 10 wherein an impedance across said array after said deposition of said lipid bilayer is at least 1 Giga-ohm for at least 4 hours and wherein said array comprises from 9 to 10,000 apertures each having a cross-sectional area of from 3 to 1.2×10⁴ nm² and spaced regularly within an area less than 2 cm².

12. The method of claim 10 further including a step of washing said surface of silicon nitride prior to said step of disposing said lipid bilayer.
